(19) United States
(12) Patent Application Publication
(10) Pub. No.: US 2023/0104350 A1
(43) Pub. Date: Apr. 6, 2023

(54) PROCESS SULFONATION OF AMINOETHYLENE SULFONIC ESTER WITH CARBON DIOXIDE ADDITION TO PRODUCE TAURINE

(71) Applicant: Archer Daniels Midland Company, Decatur, IL (US)

(72) Inventors: Chi Cheng Ma, Champaign, IL (US); James Brazdil, Leland, NC (US); Hao Luo, Decatur, IL (US)

(21) Appl. No.: 17/907,151

(22) PCT Filed: Mar. 22, 2021
(86) PCT No.: PCT/US21/23539

§ 371 Date: Sep. 23, 2022

Related U.S. Application Data
(60) Provisional application No. 62/993,305, filed on Mar. 23, 2020.

(51) Int. Cl. C07C 303/32; C07C 303/44

(52) U.S. Cl. CPC C07C 303/32; C07C 303/44

(57) ABSTRACT

A process for producing taurine, comprising mixing aminoethanol sulfate ester (AES) and a carbon dioxide, thus producing a reaction mixture, and heating the reaction mixture in the presence of a sulfite or a bisulfite, or combination thereof, such that taurine is formed.

### FIELD OF THE INVENTION

This invention relates to a process for producing taurine wherein starting material is aminoethanol sulfate ester, also called 2-aminoethyl hydrogen sulfate ester (AES).

### BACKGROUND OF THE INVENTION

Taurine, also known as 2-aminoethanesulfonic acid, is a conditional amino acid that is found in natural dietary sources, biosynthesized in the body and produced by chemical synthesis for commercial purposes. Taurine is sometimes referred to as a conditional amino acid because it is derived from cysteine like other amino acids but lacks a carboxyl group that usually belongs to amino acids. Instead, it contains a sulfonic acid group and can be called an amino sulfonic acid.

The world's annual consumption of taurine has been more than 50,000 tons, of which more than 80% are used as food and nutrition additives. Two methods have been used commercially to produce taurine, one method having ethylene oxide (EO) as starting material, and the other method having monoethanolamine (MEA) as starting material.

In the EO method, EO is reacted with sodium bisulfite to produce sodium isethionate, which is then converted via ammonolysis to sodium taurinate. Sodium taurinate is then neutralized to produce taurine. When sodium taurinate is neutralized with sulfuric acid, then a mixture of taurine and sodium sulfate is obtained. As disclosed in U.S. Pat. No. 8,609,890, sodium taurinate may be neutralized with sulfur dioxide to obtain taurine and to regenerate sodium bisulfite.

As disclosed in U.S. Pat. No. 9,145,359, the disadvantage of the EO method lies in the problematic quality of the product. More specifically, taurine produced via the EO method is a powder, and tends to form a hard cake over a short period of time during storage (in a matter of weeks), possibly due to the presence of unknown impurities. The process involves some serious hazards from the viewpoint of safety since it uses, as raw material, EO, which has extremely strong toxicity and carcinogenicity and is difficult to transport and handle. Moreover, the reaction is carried out at very high temperature (220-280° C.) and pressure (>100 bars).

In a conventional method using MEA as the starting material, taurine can be prepared by reacting MEA with sulfuric acid to obtain the intermediate 2-aminoethyl hydrogen sulfate ester (AES). The MEA method produces taurine in the form of needle crystal that has excellent stability during transportation and storage as compared to the taurine powder produced in the EO method. An added advantage of the MEA method is the mild processing conditions as compared to the high temperature and pressures as required in the EO method.

A disadvantage of the MEA method is its higher cost of manufacture and higher capital expenditures as compared to the EO method.

A disadvantage of the MEA method is the lengthy time for the sulfonation stage, typically 35-40 hours, due to the slow reaction of AES and sodium sulfite. The MEA method typically has a low product yield in the sulfonation step.

U.S. Pat. No. 9,145,359 discloses a method for the production of taurine by a cyclic process of reacting monoethanolamine, sulfuric acid, and ammonium sulfite in the presence of additives to inhibit the hydrolysis of 2-aminoethyl hydrogen sulfate (AES) intermediate. The patent states that the hydrolysis of AES is accelerated under both acidic and basic conditions. The patent states that it has now been found that the yield of taurine can be drastically increased by strictly maintaining the pH of the reaction mixture from 6.0 to 8.0 and carrying out the sulfonation reaction at a temperature of 80 to 150° C. The patent discloses examples wherein starting materials were reacted in an autoclave equipped with a stirrer for 24 hours at 110° C. under autogenous pressure for 24 hours, and examples wherein starting materials were reacted in the same autoclave for 18 hours at 120° C.

U.S. Pat. No. 10,131,621 has the same named inventor as U.S. Pat. No. 9,145,359. U.S. Pat. No. 10,131,621 discloses an extraction process for recovering aminoalcohols and glycols from aqueous streams of taurine production. The aqueous streams which contain aminoalcohols and/or glycols are first mixed with a base to increase pH and then extracted with C₃-C₆ alcohols, ketones, and ethers. The aqueous streams are then returned to their respective cyclic process for the production of taurine. The patent states that according to the MEA process disclosed in U.S. Pat. No. 9,145,359, (i) monoethanolamine is reacted first with sulfuric acid to afford 2-aminoethyl hydrogen sulfonate ester, which undergoes sulfonation reaction with ammonium sulfite to yield a mixture of taurine and ammonium sulfate, and (ii) during the sulfonation reaction, up to 15% of the intermediate ester is hydrolyzed to monoethanolamine, which is left in the waste stream as its sulfate salt, along with ammonium sulfite and ammonium sulfate, or along with sodium sulfite and sodium sulfate when sodium sulfite is used as sulfonation agent.

Typical EO and MEA methods are batch type processes that do not allow for continuous production of taurine.

It would be beneficial to have processes and products that do not have the disadvantages of conventional methods. For example, it would be beneficial to have a continuous or semi-continuous process that produces a higher yield of taurine than conventional MEA methods. It would be beneficial to have a continuous or semi-continuous process that produces taurine with reduced production of undesirable taurine by-product, e.g., N-2-Aminoethyl-2-aminoethanesulfonic acid. It would be beneficial to have a continuous or semi-continuous process that produces a high yield of taurine using less sulfite than conventional MEA methods. It would be beneficial to have a continuous process that that produces stable crystalline taurine in a shorter period of time than the batch sulfonation stage of conventional MEA methods. It would be beneficial to have a continuous process that that produces stable crystalline taurine in a shorter period of time than the batch sulfonation stage of the method disclosed in U.S. Pat. No. 9,145,359.

### BRIEF SUMMARY OF THE INVENTION

The present invention provides advantages over conventional methods and products. In an aspect, a process for producing taurine comprises mixing aminoethanol sulfate ester (AES) with a sulfite and carbon dioxide, thus producing a reaction mixture, and heating the reaction mixture such that taurine is formed. In an embodiment, the molar ratio of the sulfite or bisulfite, or combination thereof, to the aminoethanol sulfate ester (AES) in the reaction mixture is equal to or greater than 1.0 and less than about 3.0, preferably less than 2.0, more preferably less than 1.8, and even more preferably less than 1.5. In an embodiment, and the molar ratio of the carbon dioxide to the aminoethanol sulfate ester (AES) in the reaction mixture is equal to or greater than 0.1 and less than 3.0.

In an aspect, a process for producing taurine comprises adding a first stream, a second stream, and third stream to a sulfonation vessel, wherein the first stream comprises aminoethanol sulfate ester (AES), the second stream is carbon dioxide, and the third stream comprises an aqueous solution of at least one of a sulfite or a bisulfite, or combination thereof. In an aspect, the process further comprises mixing the first, second, and third streams in the sulfonation vessel, thus producing a reaction mixture. In an aspect, the process further comprises subjecting the reaction mixture to heat in the presence of an inert gas such that taurine is formed.

These and other aspects, embodiments, and associated advantages will become apparent from the following Detailed Description.

### BRIEF DESCRIPTION OF THE DRAWINGS

FIG. 1 is a process flow diagram of a continuous or semi-continuous taurine production process in accordance with aspects of the invention.

FIG. 2 depicts a drying apparatus for water removal in accordance with aspects of the invention.

FIG. 3 depicts an apparatus for sulfonation in accordance with aspects of the invention.

FIG. 4 depicts an apparatus for sulfonation in accordance with aspects of the invention.

FIG. 5 depicts a process flow diagram of a continuous or semi-continuous taurine production process in accordance with aspects of the invention.

### DETAILED DESCRIPTION

It has now been discovered that the addition of carbon dioxide to a reaction mixture of aminoethanol sulfate ester (AES) and sulfite dramatically increases taurine yield and decreases production of undesirable taurine by-product, e.g., N-2-Aminoethyl-2-aminoethanesulfonic acid depicted in formula (i) below.

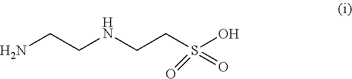

In an aspect, a continuous or semi-continuous process comprises mixing (i) aminoethanol sulfate ester, (ii) carbon dioxide, and (iii) an aqueous solution of sulfite in a sulfonation vessel, thus producing a reaction mixture, and heating the reaction mixture in the presence of an inert gas, thus converting the aminoethanol sulfate ester via sulfonation to taurine. In an embodiment, the AES and aqueous solution of sulfite is mixed together, and then the carbon dioxide is mixed with the mixed AES and aqueous solution of sulfite to produce the reaction mixture, and then the reaction mixture is conveyed into the sulfonation vessel.

The aqueous solution of sulfite may be sodium sulfite or sodium bisulfite. In an aspect, when the sulfite is bisulfite, then a base may be added to raise the pH of the reaction mixture to a range of about 7.0 to about 8.3. In an embodiment, the base is chosen from an alkali metal hydroxide (e.g., sodium hydroxide) or ammonium hydroxide, or combination thereof.

In an aspect, a process comprises continuously or semi-continuously adding a first stream, a second stream, and a third stream to a sulfonation vessel, wherein the first stream comprises aminoethanol sulfate ester (AES), wherein the second stream comprises carbon dioxide, and wherein the third stream comprises an aqueous solution of a sulfite.

In an aspect, the process comprises continuously or semi-continuously mixing the first, second and third streams in the sulfonation vessel, thus producing a mixture. In an aspect, the process comprises continuously or semi-continuously subjecting the mixture to heat, wherein the AES is converted to more taurine and less undesirable taurine by-product, e.g., N-2-Aminoethyl-2-aminoethanesulfonic acid, than the same process except for the adding of the second stream to the sulfonation vessel. In an aspect, the step of continuously or semi-continuously subjecting the mixture to heat is performed in the presence of an inert gas. In an aspect, the process further comprises subjecting the mixture to a pressure at or above autogenous pressure of the reaction mixture at reaction temperature. In an aspect, the aminoethanol sulfate ester (AES) has a residence time in the sulfonation vessel of no more than four (4) hours. In an embodiment, the aminoethanol sulfate ester (AES) has a residence time in the sulfonation vessel of no more than two (2) hours, the heat is a temperature of 110-155° C., and the mixture is subjected to a pressure of at least 100 psi. In an aspect, the sulfite is chosen from at least one of a sulfite or a bisulfite, or combination thereof, e.g., sodium sulfite, sodium bisulfite, or combination thereof. In an aspect, the process results in a taurine yield of at least 80%.

In an aspect, the adding of the carbon dioxide to the reaction mixture of AES and sulfite reduces the amount of sulfite and/or bisulfite required to obtain at least the same taurine yield in the same process except for the adding of the carbon dioxide to the reaction mixture of AES and sulfite and/or bisulfite. In an embodiment, the adding of the carbon dioxide to the reaction mixture of AES and sulfite reduces the mole ratio of sulfite to AES from about 1.8 and greater to about 1.2-1.3 to obtain at least the same taurine yield in the same process except for the adding of the carbon dioxide to the reaction mixture of AES and sulfite. In an embodiment, the adding of the carbon dioxide to the reaction mixture of AES and sulfite reduces by about 28% the mole ratio of sulfite to AES required to obtain at least the same taurine yield in the same process except for the adding of the carbon dioxide to the reaction mixture of AES and sulfite.

In an aspect, a process comprises continuously or semi-continuously adding a first stream, a second stream, and a third stream to a sulfonation vessel, wherein the first stream comprises aminoethanol sulfate ester (AES), the second stream is carbon dioxide, and the third stream comprises an aqueous solution of sulfite, wherein the first stream and the second stream are mixed in a first part of the sulfonation vessel. In an aspect, in a second part of the sulfonation vessel, the third stream is mixed with materials from the first part of the sulfonation vessel to form taurine.

In an embodiment, in addition to forming taurine, a carbamate may be formed. An example of carbamate is 2-(Carboxyamino)ethanesulfonic acid, and is depicted in formula (ii) below.

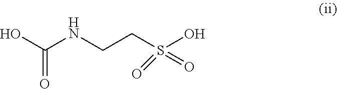

In an embodiment, the carbamate in formula (ii) may be converted to taurine with the addition of an acid, such as concentrated sulfuric acid.

In an embodiment, pH adjustment in the sulfonation vessel provides increased taurine yield and less production of undesirable taurine by-product, e.g., N-2-Aminoethyl-2-aminoethanesulfonic acid, than the same process with the exception of no pH adjustment.

The inert gas may be any suitable inert gas, including but not limited to nitrogen, helium, argon, and combinations thereof. In a preferred embodiment, the inert gas is nitrogen. In an aspect the heating step is conducted at a temperature of at least 115 ° C. and a pressure at or above autogenous pressure of the reaction mixture at reaction temperature. The presence of the inert gas subjects the mixture to the pressure greater than autogenous pressure of the reaction mixture at reaction temperature. In an aspect, the heating step is conducted at a pressure of at least 50 psi, more preferably at least 100 psi, and even more preferably at least 200 psi. In an aspect, the process results in a taurine yield of at least 80%. In an aspect, the process results in at least a 95% AES conversion to taurine. In an aspect, the aminoethanol sulfate ester has a residence time of no more than four (4) hours in the vessel. This residence time of no more than four (4) hours of the aminoethanol sulfate ester in the reaction vessel during sulfonation conversion to taurine is a substantially less than the period of time for sulfonation in conventional MEA methods. In an aspect, the aminoethanol sulfate ester has a residence time of no more than two (2) hours in the vessel.

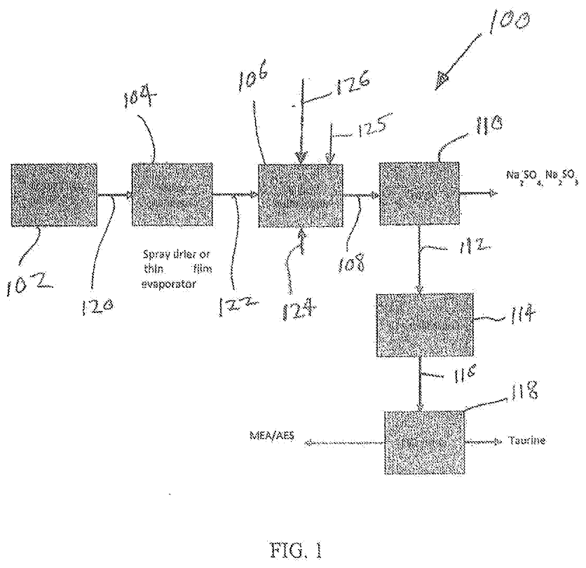

FIG. 1 is a process flow diagram of a continuous taurine production process in accordance with aspects of the invention. As shown in FIG. 1, a continuous taurine manufacturing process 100 comprises reacting step 102 wherein monoethanolamine (MEA) and sulfuric acid (H₂SO₄) are mixed and react to form the intermediate 2-aminoethyl hydrogen sulfate ester (AES). Reacting step 102 may comprise continuously conveying a stream of MEA and a stream of sulfuric acid to an AES synthesis reactor, and continuously conveying effluent 120 out of that AES synthesis reactor. Effluent 120 of reacting step 102 comprises AES and water.

Reacting step 102 is followed by water removal step 104, wherein water is removed from AES. Water removal step 104 may be performed using a spray dryer or thin film evaporator. Effluent 122 of water removal step 104 comprises AES.

After water removal step 104, effluent 122 comprising AES is then sent to a sulfonation step 106. Sulfonation step 106 comprises reacting AES with a sulfite, e.g., sodium sulfite (Na₂SO₃) and/or bisulfite to form taurine. Without being bound by theory, AES may also react with carbon dioxide to form one or more intermediates. Without being bound by theory, the one or more intermediates may react with the sulfite to form taurine. During sulfonation step 106, sodium sulfate (Na₂SO₄) may also be formed. Sulfonation step 106 may comprise using an upflow or downflow sulfonation reactor wherein effluent 122 comprising AES is continuously or semi-continuously pumped to the bottom or top of the sulfonation reactor. Similarly, a stream 124 comprising aqueous sulfite or bisulfite is continuously or semi-continuously pumped to the bottom or top of the sulfonation reactor. Similarly, stream 125 comprising carbon dioxide is continuously or semi-continuously conveyed to the bottom or top of the sulfonation reactor. In the sulfonation reactor, AES is continuously or semi-continuously mixed and reacts with sulfite and/or bisulfite, and carbon dioxide present in the sulfonation reactor. The sulfonation reactor may be sealed with a pressure head with an inert gas 126 (e.g., nitrogen gas). Sulfonation step 106 comprises continuously or semi-continuously subjecting the mixture of AES, sulfite and/or bisulfite, and carbon dioxide to heat in the presence of the inert gas. The heat may be a predetermined reaction temperature. In an aspect, the mixture of AES, sulfite or bisulfite, and carbon dioxide is continuously subjected to a pressure at or above autogenous pressure of the reaction mixture at reaction temperature. In an aspect, the pressure may be at least 200 psi inert gas (e.g., N₂). In an aspect, the heat may be at least 115° C. In an embodiment, the heat may be at least 120° C. In a preferred embodiment, the heat may be 120-155° C. In a more preferred embodiment, the heat may be 140-155° C. Effluent 108 from sulfonation step 106 comprises taurine and may also comprise Na₂SO₄ and Na₂SO₃, as well as unreacted MEA and, AES. Effluent 108 from sulfonation step 106 may also include a trace or small amount of any unreacted intermediate formed by the reaction of AES and carbon dioxide, and which did not react with sulfite and/or bisulfite to form taurine.

Effluent 108 from sulfonation step 106 may then be conveyed to chromatography step 110. In chromatography step 110, Na₂SO₄ and Na₂SO₃ is separated from taurine in effluent 108.

Effluent 112 from chromatography step 110 comprises taurine and may be conveyed to crystallization step 114. In crystallization step 114, taurine in effluent 112 is crystallized. Crystallization step may comprise cooling effluent 112 from an elevated temperature, e.g., about 100° C., to a lower temperature, e.g., about 28° C. Crystallization step 114 may be preceded by a water removal step (not shown in FIG. 1) wherein water is removed from effluent 112, e.g., by distillation, thereby concentrating the amount of taurine in effluent 112 prior to crystallization.

Effluent 116 from crystallization step 114 comprises crystallized taurine and may be conveyed to filtration step 118. In filtration step 118, crystallized taurine is separated from unreacted MEA and AES. Unreacted MEA and AES may be recycled to reaction step 102 for synthesis of AES as previously described.

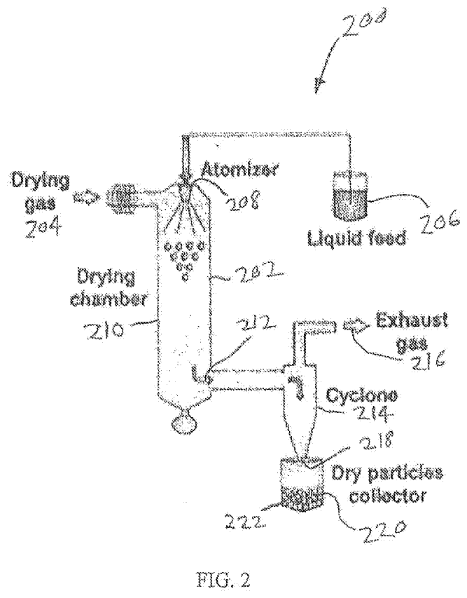

FIG. 2 depicts a drying apparatus 200 for water removal in accordance with aspects of the invention. Drying apparatus 200 comprises spray dryer 202. Drying apparatus 200 comprises drying gas 204. Drying gas 204 may be an inert gas, e.g., N₂. Liquid feed 206 may be the same as effluent 120 shown in FIG. 1. Thus, liquid feed 206 comprises AES formed in reacting step 102 shown in FIG. 1.

Spray dryer 202 may comprise drying chamber 210 and an atomizer 208 configured to atomize liquid feed 206. Effluent 212 from spray dryer 202 may be conveyed to cyclone 214. In cyclone 214, exhaust gas 216 is separated from effluent 222. Effluent 222 exits cyclone 214 through opening 218. Effluent 222, comprising AES, may be collected in a collector 220. Effluent 222 may be the same as effluent 122 shown in FIG. 1. Thus, effluent 222 comprising AES has less water than liquid feed 206.

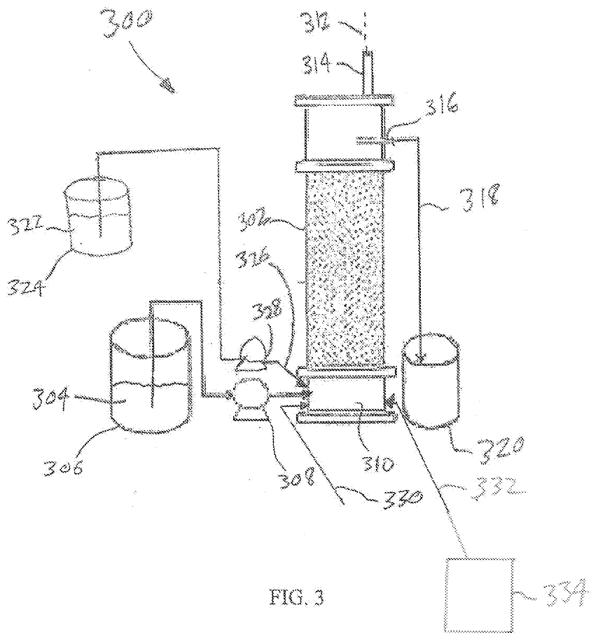

FIG. 3 depicts apparatus 300 for sulfonation step 106 shown in FIG. 1 in accordance with aspects of the invention. As shown in FIG. 3, apparatus 300 may comprise an upflow sulfonation reactor 302. Those skilled the art having the benefit of the present disclosure will recognize that the sulfonation reactor may be a downflow sulfonation reactor. Feed 304 in feed vessel 306 may be degassed by an inert gas prior to being conveyed out of feed vessel 306. The inert gas may be any suitable inert gas, including but not limited to nitrogen, helium, argon, and combinations thereof. In a preferred embodiment, the inert gas is nitrogen. Feed 304 is continuously conveyed out of feed vessel 306 by pump 308 to bottom 310 of upflow sulfonation reactor 302. Feed 304 may be the same as effluent 222 shown in FIG. 2. Thus, feed 304 comprises AES. As shown in FIG. 3, AES may be continuously pumped to the bottom of the sulfonation reactor 302. In sulfonation reactor 302, AES reacts with sulfite and/or bisulfite present in the sulfonation reactor 302 to form taurine.

Aqueous sulfite and/or bisulfite 322, e.g., aqueous sodium sulfite and/or aqueous sodium bisulfite, in vessel 324 may be degassed by an inert gas prior to being conveyed out of vessel 324. The inert gas may be any suitable inert gas, including but not limited to nitrogen, helium, argon, and combinations thereof. In a preferred embodiment, the inert gas is nitrogen. Aqueous sulfite and/or bisulfite 322 is continuously conveyed out of vessel 324 as stream 326 by pump 328 to bottom 310 of upflow sulfonation reactor 302. Stream 326 comprising aqueous sulfite and/or bisulfite 322 may be the same as stream 124 shown in FIG. 1.

Carbon dioxide 332 is continuously conveyed out of carbon dioxide source 334. Carbon dioxide source may be any suitable carbon dioxide source, e.g., a pressurized tank of carbon dioxide, a compressor conveying carbon dioxide, or a process stream of carbon dioxide. Carbon dioxide 332 may be the same as stream 125 shown in FIG. 1.

Sulfonation reactor 302 may be sealed with a pressure head with an inert gas, e.g., inert gas 330. Inert gas 330 may be the same as inert gas 126 shown in FIG. 1. Thus, sulfonation step 106 shown in FIG. 1 may be performed in apparatus 300 shown in FIG. 3. Sulfonation reactor 302 may be operated by heating the reaction mixture of AES and aqueous sulfite and/or bisulfite at a reaction temperature and under a reaction pressure, e.g., a reaction pressure of at least 200 psi inert gas (e.g., Na). Inert gas 330 subjects the reaction mixture of AES, aqueous sulfite and/or bisulfite and carbon dioxide to a pressure at or above autogenous pressure of the reaction mixture at reaction temperature. The reaction temperature may be at least 110° C. In an embodiment, the reaction temperature may be at least 120° C. In a preferred embodiment, the reaction temperature may be 120-155° C. In a more preferred embodiment, the reaction temperature may be 140-155° C. Via conduit 316, effluent 318 may be collected in vessel 320. Effluent 318 may be the same as effluent 108 shown in FIG. 1. Thus, effluent 318 comprises taurine, and may also comprise Na₂SO₄ and Na₂SO₃, as well as unreacted MEA and AES. Exhaust gas 312 comprising inert gas may exit sulfonation reactor 302 through conduit 314 as may be desired, e.g., to purge materials in sulfonation reactor, or maintain a predetermined pressure in the sulfonation reactor 302.

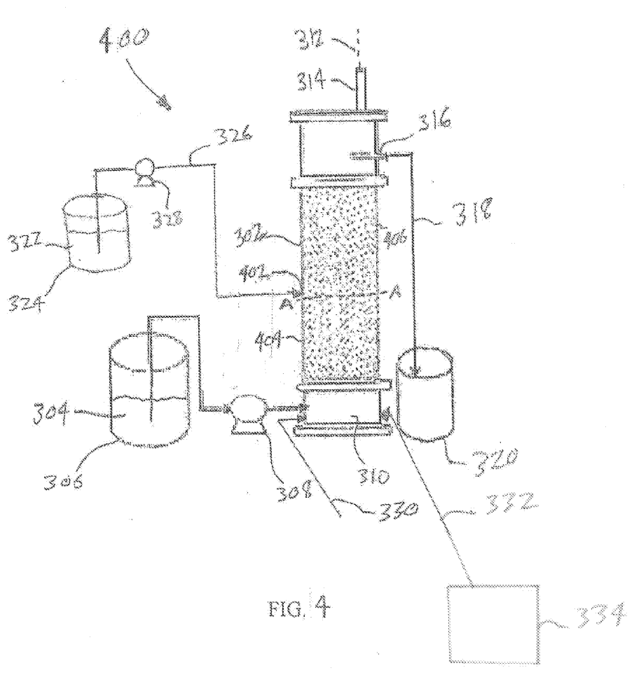

FIG. 4 depicts apparatus 400. Apparatus 400 is the same as apparatus 300 shown FIG. 3, with the exception that aqueous sulfite and/or bisulfite stream 326 is continuously conveyed by pump 328 to upper part 406 of upflow sulfonation reactor 302 through inlet 402, rather than to bottom 310 of upflow sulfonation reactor 302. Without being bound by theory, AES of feed 304 may react with carbon dioxide 332 to form one or more intermediates in lower part 404 of upflow sulfonation reactor 302. As shown in FIG. 4, lower part 404 is below dashed line A-A and upper part 406 is above dashed line A-A. In upper part 406, aqueous sulfite and/or bisulfite of stream 326 reacts with materials from lower part 404 to form taurine, without production of undesirable taurine by-product.

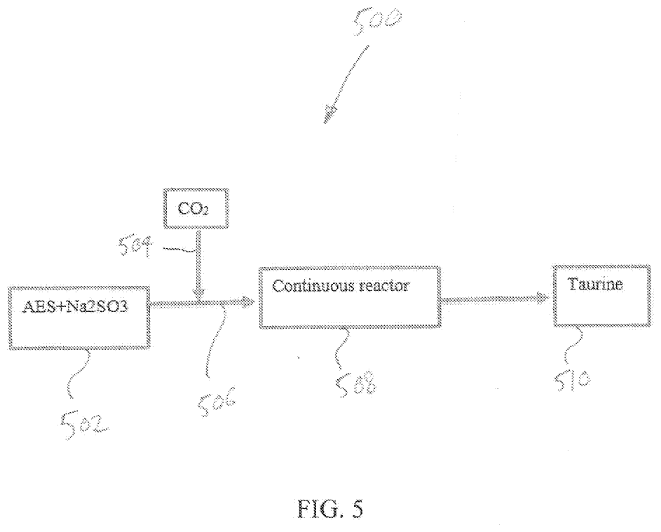

FIG. 5 depicts a process flow diagram of a continuous or semi-continuous taurine production process in accordance with aspects of the invention. As shown in FIG. 5, a continuous taurine manufacturing process 500 comprises mixing mixture 502 of AES and a sulfite, e.g., sodium sulfite, with stream 504 of carbon dioxide to form mixture 506. Mixture 506 is conveyed to continuous reactor 508 wherein taurine 510 is formed. The AES in mixture 502 may be formed as previously described with respect to FIG. 1 and FIG. 2. Thus, the AES in mixture 502 may be the same as effluent 122 in FIG. 1, and effluent 222 in FIG. 2. Continuous reactor 508 may be the same as sulfonation reactor 302 in FIG. 3 and FIG. 4. Continuous reactor 508 may be an upflow reactor or a downflow reactor. An inert gas, like insert gas 330 shown in FIG. 3, may be conveyed to continuous reactor 508. Due the presence of the insert gas, mixture 506 is subjected to a pressure at or above autogenous pressure of the reaction mixture 506 at reaction temperature in continuous reactor 508.

The following example further describes taurine synthesis in accordance with aspects of the present invention.

### Example 1

9.65 g 40wt % sodium bisulfite (37 mmol) (SBS) solution was added to a 75 mL Hastelloy Parr reactor. Equal molar amounts of NaOH was dissolved in 16.00 g H₂O, and then added to SBS solution. 4.30 gram of AES (SBS/AES=1.3) was measured and added to the mixture. The system was purged with N₂ for three times, then added 200 psi CO₂. The initial pH value of the feed was recorded, and a feed sample was taken for analysis using ¹H NMR and ¹³C NMR. The reactions were performed at stirring speed 1000 rpm, temperature 150° C. for 1.5 hours. After reaction, the reactor was cooled down to room temperature, and the product mixture analyzed by ¹H NMR (Table 1) with and ¹³C NMR. In this example, the molar ratio of the carbon dioxide to the AES in the reaction mixture about 1.0 as shown in the following calculations. 4.30 g AES/141.14 g/mol=0.0305 mol; Moles CO₂=(13.61 atm)(0.075L)/(0.08207 L atm/deg mol)(298.15 K)=0.042 mol; Molar ratio of carbon dioxide to AES is 0.042 mol/0.0305 mol, or about 1.4.

TABLE 1
NMR analysis of taurine and other products

| | | | | 2-(Carboxy | N-2- | |
|---|---|---|---|---|---|---|
| | | | | amino)ethane | Aminoethyl-2- | |
| | | | Taurine | sulfonic acid | aminoethane | 2-oxazolidinone |
| | | MEA | yield | yield | sulfonic acid | yield |
| | AES | (molar) | (molar) | (molar) | (molar) | (molar) |
| Product | 0% | 20% | 58% | 0% | <1% | 20% |

As shown in Table 1, 0% AES was present in the product mixture, thus indicating that all AES present in the reactor was reacted. As shown in Table 1, the product mixture was 58% of taurine yield (molar), 20% MEA (molar), 20% 2-oxazolidinone (molar), and less than 1% undesirable taurine by-product, i.e., N-2-Aminoethyl-2-aminoethane sulfonic acid (molar). The MEA and 2-oxazolidinone in the product mixture may be recycled for upstream processing to yield more taurine. For example, MEA can be recycled back to reacting step 102 of FIG. 1 for making AES from MEA. The 2-oxazolidinone in the product mixture may be recycled to the sulfonation reactor for production of taurine. The same method but without the carbon dioxide addition in accordance with the present invention would have substantially greater than the less than 1% undesirable taurine by-product, i.e., N-2-Aminoethyl-2-aminoethane sulfonic acid (molar). Those skilled in the art having the benefit of the present invention will recognize that the carbon dioxide addition disclosed herein is an important and valuable benefit over the same method devoid of the carbon dioxide addition.

Those skilled in the art having the benefit of the present disclosure will recognize that the above features disclosed herein improve upon conventional MEA processes by reducing the time of sulfonation in conventional MEA processes by a factor of at least four. Those having skill in the art, with the knowledge gained from the present disclosure, will recognize that various changes can be made to the disclosed processes in attaining these and other advantages, without departing from the scope of the present disclosure. As such, it should be understood that the features of the disclosure are susceptible to modifications and/or substitutions. The specific embodiments illustrated and described herein are for illustrative purposes only, and not limiting of the invention as set forth in the appended claims.

In the claims:

1. A process for producing taurine comprising:
mixing aminoethanol sulfate ester (AES) with carbon dioxide thus producing a reaction mixture; and
heating the reaction mixture in the presence of a sulfite or a bisulfite, or combination thereof, such that taurine is formed.

2. The process of claim 1, wherein the sulfite or bisulfite is present in the reaction mixture.

3. The process of claim 1, wherein a base is present in the reaction mixture.

4. The process of claim 3, wherein the base is chosen from an alkali metal hydroxide and ammonium hydroxide, and a combination thereof.

5. The process of claim 4, wherein the alkali metal hydroxide is sodium hydroxide.

6. The process of claim 1, wherein the mixing comprises mixing the aminoethanol sulfate ester (AES) with carbon dioxide, and with the sulfite or bisulfite, or combination thereof, thus producing the reaction mixture.

7. The process of claim 6, wherein the mixing comprises mixing a base, the aminoethanol sulfate ester (AES), the carbon dioxide, and the sulfite or bisulfite, or combination sulfite or bisulfite, thus producing the reaction mixture.

8. The process of claim 6, wherein the molar ratio of the sulfite or bisulfite, or combination thereof, to the aminoethanol sulfate ester (AES) in the reaction mixture is equal to or greater than 1.0 and less than about 3.0, and the molar ratio of the carbon dioxide to the aminoethanol sulfate ester (AES) in the reaction mixture is equal to or greater than 0.1 and less than 3.0.

9. The process of claim 8, wherein the molar ratio of the sulfite or bisulfite, or combination thereof, to the aminoethanol sulfate ester (AES) in the reaction mixture is equal to or greater than 1.0 and less than 2.0.

10. The process of claim 8, wherein the molar ratio of the sulfite or bisulfite, or combination thereof, to the aminoethanol sulfate ester (AES) in the reaction mixture is equal to or greater than 1.0 and less than 1.8.

11. The process of claim 8, wherein the molar ratio of the sulfite or bisulfite, or combination thereof, to the aminoethanol sulfate ester (AES) in the reaction mixture is equal to or greater than 1.0 and less than 1.5.

12. The process of claim 1, wherein the sulfite or bisulfite, or combination thereof, is added to the reaction mixture before or during the step of heating the reaction mixture.

13. The process of claim 1, wherein the step of heating is performed in the presence of an inert gas.

14. A process for producing taurine, comprising:
a) adding a first stream, a second stream, and third stream to a sulfonation vessel, wherein the first stream comprises aminoethanol sulfate ester (AES) and the second stream is carbon dioxide, and the third stream comprises an aqueous solution of at least one of a sulfite or a bisulfite, or combination thereof;
b) mixing the first, second and third streams in the sulfonation vessel, thus producing a reaction mixture; and
c) subjecting the reaction mixture to heat in the presence of an inert gas such that taurine is formed.

15. The process of claim 14, wherein the molar ratio of the sulfite to the aminoethanol sulfate ester (AES) in the reaction mixture is equal to or greater than 1.0 and less than about 3.0, and the molar ratio of the carbon dioxide to the aminoethanol sulfate ester (AES) in the reaction mixture is equal to or greater than 0.1 and less than 3.0.

16. The process of claim 14, wherein the first stream and the second stream are mixed in a first part of the sulfonation vessel, wherein materials from the first part of the sulfonation vessel are mixed and react with the third stream in a second part of the sulfonation vessel to form to taurine.

17. The process of claim 14, wherein the aminoethanol sulfate ester (AES) has a residence time of no more than four hours in the sulfonation vessel.

18. The process of claim 14, wherein the aminoethanol sulfate ester (AES) has a residence time of no more than two hours in the sulfonation vessel.

19. The process of claim 14, wherein the inert gas is chosen from nitrogen, argon, helium, and combinations thereof.

20. The process of claim 14, wherein the inert gas is nitrogen.

21-48. (canceled)
